(19) United States
(12) Patent Application Publication
(10) Pub. No.: US 2011/0009649 A1
(43) Pub. Date: Jan. 13, 2011

(54) METHOD FOR PREPARING 2-(n-BUTYL)-5-NITROBENZOFURAN

(75) Inventors: Thierry Durand, Tassin La Demi Lune (FR); Ousmanne Diouf, Vienne (FR); Stephane Lemeune, Lyon (FR); Jean-Francois Marcoux, Communay (FR); Vivien Henryon, Lyon (FR); Jerome Monbrun, Miramas (FR); Madeleine Delamare, Lyon (FR)

Correspondence Address: ROTHWELL, FIGG, ERNST & MANBECK, P.C., 1425 K STREET, N.W., SUITE 800, WASHINGTON, DC, 20005, US

(73) Assignee: FINORGA

(21) Appl. No.: 12/594,861

(22) PCT Filed: Apr. 4, 2008
(86) PCT No.: PCT/FR2008/000471

§ 371 Date: Sep. 29, 2010

(30) Foreign Application Priority Data
Apr. 6, 2007 (FR) 0702543

(51) Int. Cl. C07D 307/79; C07C 209/22

(52) U.S. Cl. 549/469; 568/706

(57) ABSTRACT

A method for preparing 2-(n-butyl)-5-nitrobenzofuran from 1-halo-4-nitrobenzene, characterized in that 1-hexen-3-ol is reacted, and then the product is subject to a Claisen rearrangement, followed by catalytic intramolecular cyclization.

The present invention relates to a method for preparing 2-(n-butyl)-5-nitrobenzofuran of formula:

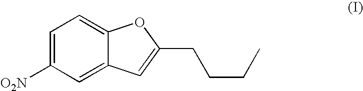

notably useful as an intermediate for preparing dronedarone or one of its salts.

Dronedarone as well as its salts was described in European patent application EP 471609. This product is particularly interesting as an anti-arrhythmic agent and finds application in the cardiovascular field and notably for preventing certain types of mortality after myocardial infarctions.

According to European patent application EP 471609, preparation of dronedarone is carried out via 2-(n-butyl)-5-nitrobenzofuran. Preparation of 2-(n-butyl)-5-nitrobenzofuran is performed starting from 2-hydroxy-5-nitrobromobenzyl and applies a reaction using triphenylphosphine for preparing 2-hydroxy-5-nitrobenzyltriphenylphosphonium bromide. However, it was necessary to find a performing industrial method avoiding the use of 2-hydroxy-5-nitrobromobenzyl, a costly precursor and allowing access to this intermediate molecule, the preparation of which generated a large amount of waste, in particular triphenylphosphine oxide.

Patents WO-A 01/28974 and WO-A 01/29019 describe a method for preparing 5-nitrobenzofuran from salicylic aldehyde and including 4 steps via intermediates like 2-(2-formyl-4-nitrophenoxy)carboxylic acid. However the use of salicylic acid is costly and the presence of aldehyde functions on the intermediates makes the method sensitive to oxidation.

In patent EP-A 1 116 719, a 5-nitro-2(3H)-benzofuranone is applied in the presence of pentanoic acid and pentanoic anhydride in order to lead to 3-(1-hydroxypentylidene)-5-nitro-2(3H)-benzofuranone and subsequently in an acid medium to 2-(n-butyl)-5-nitrobenzofuran. Such a method using 5-nitro-2(3H)-benzofuranone as a precursor is not very economical from an industrial point of view.

Patent U.S. Pat. No. 6,984,741 describes the synthesis of 2-(n-butyl)-5-nitrobenzofuran from methyl salicylate and from methyl 2-bromohexanoate in 7 steps via the intermediate 2-(n-butyl)-5-nitro-3(2H)-benzofuranone. This method in 7 steps is long and not very economical.

The object of the present invention relates to obtaining 2-(n-butyl)-5-nitrobenzofuran in 3 steps from 1-halo-4-nitrobenzene, by reaction with 1-hexen-3-ol, followed by a Claisen rearrangement and then by catalyzed intramolecular cyclization.

It is now possible to obtain 2-(n-butyl)-5-nitrobenzofuran by an innovating approach having a real industrial benefit, because of its selectivity and of the high yields which may be obtained.

According to the invention:
a) 1-hexen-3-ol is reacted on 1-halo-4-nitrobenzene after deprotonation of the alcohol in a basic medium,
b) the obtained 4-nitrophenyl-1-vinylbutylether, of formula

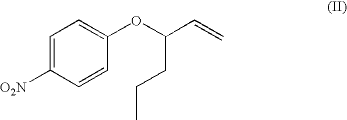

is subject to a Claisen rearrangement in order to obtain the 2-(hex-2-en-1-yl)-4-nitrophenol of formula

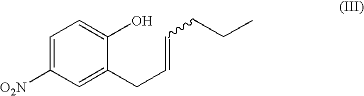

and then
c) the 2-(hex-2-en-1-yl)-4-nitrophenol is subject to intramolecular cyclization catalyzed by metals from the platinum group, into 2-(n-butyl)-5-nitrobenzofuran of formula (I).

According to the invention, the halogen of 1-halo-4-nitrobenzene is advantageously selected from fluorine or chlorine.

Step a) consists in the preparation of 4-nitrophenyl-1-vinylbutylether.

According to the invention, step a) of the reaction of 1-hexen-3-ol on 1-halo-4-nitrobenzene, is carried out according to known methods which do not alter the remainder of the molecule, notably after deprotonation of the alcohol in the presence of a mineral or organic base, in a homogeneous or heterogeneous medium, preferably in the presence of an alkaline hydride (for example sodium hydride), or of an alkaline carbonate (for example sodium or potassium carbonate). Deprotonation and the reaction with 1-halo-4-nitrobenzene are carried out in a polar aprotic solvent such as an amide (dimethylformamide for example) or a nitrile, at a temperature comprised between 20° C. and the reflux temperature of the reaction mixture. According to the invention, the step b) for Claisen rearrangement of 4-nitrophenyl-1-vinylbutylether into 2-(hex-2-en-1-yl)-4-nitrophenol, is achieved by thermal activation or in the presence of catalysts, it is notably achieved by heating at temperatures above 100° C., with or without any solvent. The rearrangement is applied in the presence of a protic polar solvent (notably in a hydro-alcoholic medium, for example in an ethanol-water medium), or in the presence of an aprotic but not very polar solvent or an aprotic apolar solvent, notably in ethers, for example di-isopropylether or diphenylether), in hydrocarbons or halogenated solvents (for example o-dichlorobenzene or trichlorobenzene) or in the presence of a mixture of the aforementioned solvents, at a temperature comprised between 100 and 260° C., and more particularly between 150 and 180° C.

2-(hex-2-en-1-yl)-4-nitrophenol of formula (III) is a novel product which also enters the scope of the present invention.

According to the invention, the step c) for catalytic intramolecular cyclization of 2-(hex-2-en-1-yl)-4-nitrophenol into 2-(n-butyl)-5-nitrobenzofuran is carried out starting from the nitrophenol derivative of formula (III) with a catalytic method, in the presence of metals from the platinum group, preferentially palladium and more particularly salts of palladium^{II}, either in the presence or not of an organic or mineral base or of an organic or mineral oxidant (notably in the presence of dissolved oxygen).

More particularly, the operation is performed in the presence of a Pd^{II} salt (such as notably halides or carboxylates, notably palladium chloride and acetate) either liganded (for example with phosphines or nitriles, preferentially bis(benzonitrile)-palladium(II) chloride [PdCl₂(PhCN)₂] or bis(acetonitrile)-palladium(II) chloride [PdCl₂(MeCN)₂]) or non-liganded.

If necessary, the base is advantageously selected from alkaline salts such as alkaline carbonates or bicarbonates or carboxylates (sodium or potassium carbonate, sodium or potassium bicarbonate for example, sodium acetate for example) or organic bases such as the nitrogen-containing bases (triethylamine for example), and the reaction is conducted in the presence of an organic oxidant (such as benzoquinone), a mineral agent such as copper salts (copper acetate for example) or in the presence of a oxidant gas (dissolved oxygen), in an either aprotic but not very polar or aprotic apolar organic solvent such as an ether (dioxane, tetrahydrofuran for example) or in an aromatic hydrocarbon (xylene for example) or in an aprotic polar solvent (such as acetonitrile for example) or in a mixture of the aforementioned solvents, at a temperature comprised between 20° C. and the reflux temperature of the reaction mixture.

Preferably, the catalytic amounts are comprised between 0.1 and 1 equivalent.

Preferably, the operation is performed in the presence of bis(benzonitrile)-palladium(II) chloride in the presence of an organic oxidant such as benzoquinone, and of sodium carbonate.

The products obtained according to steps a) to c) may be purified by chromatography.

The present invention is particularly of interest because of its steps for Claisen rearrangement and intramolecular cyclization, which are particularly selective and lead to high yields. Notably, cyclization of 2-(hex-2-en-1-yl)-4-nitrophenol rapidly and selectively leads to 2-(n-butyl)-5-nitrobenzofuran without any formation of chromene, under mild catalytic conditions.

Dronedarone may be obtained from 2-(n-butyl)-5-nitrobenzofuran for example according to the method described in European patent application EP 471609.

The following examples given as non-limiting examples, illustrate the present invention.

### EXAMPLE A

### Preparation of 4-nitrophenyl-1-vinylbutylether

Into a 250 ml three-neck flask, equipped with a magnetized bar, placed on a magnetic stirrer, are successively introduced sodium hydride (46.8 mmol) and anhydrous dimethylformamide (60 ml) (grey suspension).

A solution of 1-hexen-3-ol (42.5 mmol) in anhydrous dimethylformamide (60 ml) placed in a 100 ml dropping funnel is added dropwise. Exothermy of the reaction is controlled by a cold water bath (20° C.) so that the temperature of the mixture does not exceed 30° C. It is maintained under stirring for 2 hours 20 min at 26° C. (gas evolvement ceases after 1 hour of reaction).

At 26° C., a solution of 1-fluoro-4-nitrobenzene (28.4 mmol) in dimethylformamide (12 ml) is added with a flow rate of 0.5 ml/min. Addition is accompanied by slight exothermy (+4° C.). The mixture is maintained for 3 hours 30 min at 26° C., under stirring.

The raw reaction mixture is slowly poured over an aqueous solution saturated with ammonium chloride under stirring; exothermy is controlled by an ice water bath (5° C.) so that the temperature of the mixture does not exceed 30° C. Stirring is maintained for 15 further minutes.

Extraction is carried out with 5×150 ml of diethylether, and the organic phase is then dried on sodium sulfate, filtered and dry concentrated in the rotary evaporator.

7.94 g of raw 4-nitrophenyl-1-vinylbutylether as an orange-colored oil are thereby obtained:

Conversion rate of 1-fluoro-4-nitrobenzene=100%.

Purification is carried out by chromatography on 40-63 μm silica gel (150 g), heptane/AcOEt gradient 100/0 (500 ml)→95/5 (1 liter), by collecting the fractions: Rf=0.63 (Heptane/AcOEt 75/25) and then by dry concentrating them.

Thus, 5.85 g of purified 4-nitrophenyl-1-vinylbutylether are obtained as a yellow-orange-colored oil:

Isolated yield=93%
Titer>95% (estimated by ¹H NMR).

¹H NMR 250 MHz (CDCl₃), δ in ppm: 0.96 (3H, t, CH₂CH₃), 1.47 (2H, m, CH₂CH₃), 1.74 (2H, m, CHCH₂CH₂), 4.71 (1H, td, O—CHCH₂), 5.23 (1H, m, HC═CHCH—O), 5.28 (1H, m, HC═CHCH—O), 5.82 (1H, m, C═CHCH—O), 6.94 (2H, d, O—C═CHₐᵣ), 8.15 (2H, d, O₂N—C═CHₐᵣ).

### EXAMPLE B

### Preparation of (cis and trans) 2-(hex-2-en-1-yl)-4-nitrophenol

Into a 25 ml stainless steel reactor, equipped with a magnetized bar, placed on a magnetic stirrer, are successively introduced 2.0 g of 4-nitrophenyl-1-vinylbutylether (9.04 mmol) and 2 v of an EtOH/H₂O mixture (70/30) (2.8 ml/1.2 ml).

The whole is heated to 180° C. under stirring (autogenous pressure: 9 bars). After 3 hours of reaction, the reaction mixture is cooled to 26° C. The raw mixture is dry concentrated by means of a rotary evaporator.

1.64 g of raw (cis and trans) 2-(hex-2-en-1-yl)-4-nitrophenol) are obtained as a dark brown oil:

Conversion rate of 4-nitrophenyl-1-vinylbutylether=100% (TLC and HPLC)
Isolated yield=82%.

Purification is carried out on 40-63 μm silica gel (85 g), heptane/AcOEt gradient 90/10 (200 ml)→80/20 (300 ml)→70/30 (500 ml), by collecting the fractions: Rf=0.33 (Heptane/AcOEt 75/25) and then by dry concentration.

Thus, 1.52 g of (cis et trans) 2-(hex-2-en-1-yl)-4-nitrophenol are obtained as a beige solid:

Isolated yield of (cis et trans) 2-(hex-2-en-1-yl)-4-nitrophenol=78%
Titer>95% (estimated by ¹H NMR)

### M.p.=58.1-60.5° C.

¹H NMR 250 MHz (CDCl₃), δ in ppm: 0.91 (3H, t, CH₃), 1.42 (2H, m, CH₂CH₃), 2.05 (2H, m, ═CHCH₂CH₂), 3.42 (2H, d, CarCH₂CH═), 5.65 (2H, m, —HC═CH—), 6.22 (1H, s, —OH), 6.89 (1H, d, O—C═CHar), 8.05 (2H, m, O₂N—C═CHarCHar, O₂N—C═CHarCq).

### EXAMPLE C

### Cyclization into Benzofuran (1 Equivalent of Catalyst)

Into a 50 ml three-neck flask, equipped with a magnetized bar, placed on a magnetic stirrer, are successively introduced 100 mg (0.452 mmol) of 2-(hex-2-en-1-yl)-4-nitrophenol, 173 mg (0.452 mmol) of bis(benzonitrile)-palladium(II) chloride [PdCl₂(PhCN)₂], 48 mg (0.452 mmol) of sodium carbonate, 49 mg of 1,4-benzoquinone and 30 ml of 1,4-dioxane.

The mixture is stirred and then heated to 80° C. for 3 hours. The mixture is then filtered, dry concentrated and then taken up with 2 ml of dioxane and added with 1 ml of deionized water and extracted with 4×5 ml of dichloromethane. The organic phase is dried on sodium sulfate, filtered and dry concentrated (precipitation of Pd(0)). The residue is taken up with minimum toluene (orange-colored solution, presence of black solid).

Purification is carried out by chromatography on 15-25 μm silica gel (8 g), pure toluene (100 ml), by collecting the fractions: Rf=0.76 (toluene 100%) and then by dry concentrating them.

50 mg of 2-butyl-nitrobenzofuran are obtained as a slightly yellow oil.

Isolated yield of 2-butyl-nitrobenzofuran=50%
Titer>90% (estimated by ¹H NMR)

¹H NMR, 250 MHz, (CDCl₃), δ in ppm: 0.97 (3H, t, CH₃), 1.43 (2H, m, CH₂CH₂CH₃), 1.75 (2H, m, CH₂CH₂CH₂), 2.81 (2H, t, C═C(O)CH₂CH₂), 6.51 (1H, s, O—C═CH—Car), 7.45 (1H, d, O—C═CHar-C), 8.14 (1H, dd, O₂N—C═CHarCHar), 8.39 (1H, d, O₂N—C═CHarCq)

### EXAMPLE D

### Cyclization into Benzofuran (0.1 Equivalent of Catalyst)

Into a 8 ml pill-making machine, equipped with a magnetized bar, placed on a heating magnetic stirrer, are introduced 8.7 mg (0.023 mmol) of bis(benzonitrile)-palladium(II) chloride [PdCl₂(PhCN)₂], 24 mg (0.225 mmol) of sodium carbonate, 25 mg (0.225 mmol) of 1,4-benzoquinone and 4 ml of 1,4-dioxane. The mixture is stirred and heated to 60° C.: mixture A

A solution of 50 mg (0.225 mmol) of 2-(hex-2-en-1-yl)-4-nitrophenol in 1 ml of 1,4-dioxane is added within 1 hour onto the mixture A.

The whole is maintained for 1 hour at 60° C. under stirring after completing the addition.

Chemical yield of 2-butyl-nitrobenzofuran=85% (assayed by HPLC)
Conversion rate=99% (assayed by HPLC).

1. A method for preparing 2-(n-butyl)-5-nitrobenzofuran from 1-fluoro-4-nitrobenzene, characterized in that 1-hexen-3-ol is reacted, and the product is then subject to a Claisen rearrangement, followed by catalyzed intramolecular cyclization.

2. The preparation method according to claim 1, characterized in that:
a) the 1-hexen-3-ol is reacted on 1-halo-4-nitrobenzene after deprotonation of the alcohol in a basic medium,
b) the 4-nitrophenyl-1-vinylbutylether of formula: 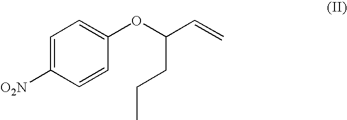
is subject to a Claisen rearrangement in order to obtain the 2-(hex-2-en-1-yl)-4-nitrophenol of formula: 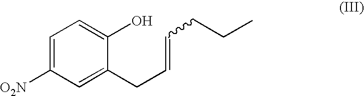
and then
c) the 2-(hex-2-en-1-yl)-4-nitrophenol is subject to intramolecular cyclization catalyzed by metals of the platinum group, into 2-(n-butyl)-5-nitrobenzofuran of formula (I).

3. The preparation method according to claim 1, characterized in that the reaction of 1-hexen-3-ol on 1-fluoro-4-nitrobenzene, or on 1-chloro-4-nitrobenzene, is carried out in the presence of a mineral or organic base, selected from an alkaline hydride or an alkaline carbonate.

4. The preparation method according to claim 1, characterized in that the Claisen rearrangement is carried out by heating to temperatures above 100° C., with or without any solvent.

5. The preparation method according to claim 4, characterized in that the Claisen rearrangement is carried out in the presence of a solvent selected from a hydro-alcoholic mixture, an ether, a hydrocarbon or a halogenated solvent or in the presence of a mixture of these solvents, at temperatures comprised between 100 and 260° C.

6. The method for preparing 2-(n-butyl)-5-nitrobenzofuran from 2-(hex-2-en-1-yl)-4-nitrophenol, claim 1, characterized in that the intramolecular cyclization is catalyzed by palladium.

7. The method for preparing 2-(n-butyl)-5-nitrobenzofuran from 2-(hex-2-en-1-yl)-4-nitrophenol, claim 1, characterized in that catalytic intermolecular cyclization is carried out, either in the presence or not of an organic or mineral base, and of an organic or mineral oxidant.

8. The preparation method according to claim 6, characterized in that the catalytic intramolecular cyclization is carried out in the presence of a Pd^{II} salt.

9. The preparation method according to claim 8, characterized in that the Pd^{II} salt is selected from palladium halides or carboxylates either liganded with phosphines or nitriles or non-liganded.

10. The preparation method according to claim 9, characterized in that the Pd^{II} salt is selected from palladium chloride or acetate either liganded or non-liganded, preferably bis(benzonitrile)-palladium(II) chloride [PdCl₂(PhCN)₂], or bis(acetonitrile)-palladium(II) chloride [PdCl₂(MeCN)₂].

11. The preparation method according to claim 7, characterized in that the oxidant is selected from benzoquinone, copper salts or an oxidant gas.

12. The preparation method according to claim 7, characterized in that the base is selected from alkaline salts or organic bases.

13. The preparation method according to claim 12, characterized in that the alkaline salts or the organic bases are selected from alkaline carbonates or bicarbonates or carboxylates or from nitrogen-containing bases.

14. 2-(hex-2-en-1-yl)-4-nitrophenol of formula: [chemical structure]
